(19) United States
(12) Patent Application Publication
(10) Pub. No.: US 2023/0201417 A1
(43) Pub. Date: Jun. 29, 2023

(54) Pharmaceutical composition for eardrum repair and use thereof

(71) Applicant: Taipeibio Co., Ltd., Taipei City (TW)

(72) Inventors: Wen-Fu Lai, Taipei City (TW); Li-Hsuan Chiu, Taipei City (TW); Fu-Chen Kung, Taipei City (TW)

(21) Appl. No.: 17/562,452

(22) Filed: Dec. 27, 2021

(51) Int. Cl. A61L 27/26; A61L 27/54; A61K 38/39

(52) U.S. Cl. CPC A61L 27/26; A61L 27/54; A61K 38/39; A61L 2430/14

(57) ABSTRACT

A pharmaceutical composition for eardrum repair includes: collagen present in an amount of 8 wt % to 12 wt %; a forming agent present in an amount of 19 wt % to 22 wt %; and rest of a solvent, wherein the forming agent is a polymer of polyethylene oxide (PEO) and polypropylene oxide (PPO), polystyrene, polyethylene, polypropylene, polymethylmethacrylate, poly(N-isopropylacrylamide), poly[2-(dimethylamino)ethyl methacrylate] (pDMAEMA) hydroxypropylcellulose, poly(vinylcaprolactame), poly-2-isopropyl-2-oxazoline, polyvinyl methyl ether or a combination thereof.

### BACKGROUND

### 1. Field

The present disclosure relates to a pharmaceutical composition for eardrum repair and a use thereof.

### 2. Description of Related Art

The healing of eardrum rupture is related to the size of the defect area. The natural healing rate of small defects can reach more than 80% after being treated with medicine and carefully taken care of. If the defect exceeds one-third of the eardrum area, the natural healing rate drops to less than 20%. For eardrum defects that cannot be healed naturally, transplantation is carried out by creating incisions behind the ears. Alternatively, the minimally invasive surgery for eardrum repair may be used, there is no need to incise the ear, but the eardrum and ossicles are directly repaired from the ear canal by using an endoscope. However, since endoscopic surgery is performed in a smaller ear canal space and under a 2D screen, the doctor is required to judge the anatomical distance and relative position of the ear canal space. Most of the time, doctors operate with one hand and stare at the screen, which is actually a laborious and vision-damaging operation. If the patient's ear canal is too small or too curved, it will take more time to enlarge the ear canal if necessary.

Therefore, a novel therapeutic manner is demanded, wherein the ruptured eardrums of patients can be repaired without surgery.

### SUMMARY

The present disclosure provides a pharmaceutical composition for eardrum repair, which comprises: collagen present in an amount of about 8 wt % to 12 wt %; a forming agent present in an amount of about 19 wt % to 22 wt %; and rest of a solvent, wherein the forming agent is a polymer of polyethylene oxide (PEO) and polypropylene oxide (PPO), polystyrene, polyethylene, polypropylene, polymethylmethacrylate, poly(N-isopropylacrylamide), poly[2-(dimethylamino)ethyl methacrylate] (pDMAEMA) hydroxypropylcellulose, poly(vinylcaprolactame), poly-2-isopropyl-2-oxazoline, polyvinyl methyl ether or a combination thereof.

The present disclosure further provides a method for repairing eardrum of a subject, wherein the method comprises a step of: administering to a subject in need thereof the aforesaid pharmaceutical composition.

The pharmaceutical composition of the present disclosure is a tissue regeneration material. When the pharmaceutical composition is administered to the damaged or ruptured eardrum, the pharmaceutical composition can self-polymerize to form the collagen membrane as the cell growth scaffold, so the eardrum cells can attach to the formed scaffold and grow rapidly. In addition, the formed scaffold can simulate the structure of the eardrum, construct an environment for epithelial cell growth, and promote epithelial cell differentiation and regeneration. The formed scaffold can also provide a growth environment for mucosal tissues, regenerate the inner membrane layer of the eardrum, and generate new healing tissue similar to the original tissue. In addition, the pharmaceutical composition can rapidly form a solidified membrane structure with suitable strength after contacting the eardrum, and at the same time provide a cell growth scaffold and isolation function. Thus, the subsequent infections of the inner ear caused by pathogen invasion can be prevented, and the damaged or ruptured eardrums of the patients can be repaired without complicated operations.

The pharmaceutical composition of the present disclosure comprising suitable amounts of collagen and forming agent can maintain better fluidity when operating in a wider ambient temperature. In addition, the pharmaceutical composition of the present disclosure has a more precise thermo-transition temperature frame and faster thermo-gelation rate. When the pharmaceutical composition of the present disclosure is administered to the eardrum to be repaired, effective gelation and adhesion of the pharmaceutical composition can be achieved and therefore, more advantageous emergency rescue and tissue regeneration can be realized.

In some embodiments, the pharmaceutical composition may have a thermo-transition temperature near to the ear canal temperature. For example, the pharmaceutical composition may have a thermo-transition temperature ranging from 32° C. to 37° C. When the pharmaceutical composition has suitable thermo-transition temperature, the pharmaceutical composition can be converted from a fluid into a gel or solid at the environment where the pharmaceutical composition is administered, for example, the ear canal.

In some embodiments, the pharmaceutical composition may have a gelation time less than 120 seconds, wherein the gelation time may depend upon the amounts of the collagen and the forming agent. For example, the gelation time may be in a range from 10 seconds to 120 seconds, 20 seconds to 120 seconds, 30 seconds to 120 seconds, 30 seconds to 110 seconds, 40 seconds to 110 seconds, 40 seconds to 100 seconds, 40 seconds to 90 seconds, 50 seconds to 90 seconds or 50 seconds to 80 seconds. When the pharmaceutical composition has the suitable gelation time, the user convenience can be improved.

In some embodiments, the pharmaceutical composition may comprise about 8 wt % to 12 wt % of collagen, for example, 8 wt %, 8.5 wt %, 9 wt %, 9.5 wt %, 10 wt %, 10.5 wt %, 11 wt %, 11.5 wt % or 12 wt %. In some embodiment, the pharmaceutical composition may comprise about 9.5 wt % to 11.5 wt % of collagen. In some embodiments, the pharmaceutical composition may comprise about 10 wt % of collagen.

In some embodiments, the collagen used herein may be unmodified collagen, modified collagen, reconstituted collagen (R-collagen) or a combination thereof. The collagen may facilitate the tissue repair and/or cell growth. The species for collagen is not particularly limited, and the collagen may be derived from any animals known in the art. For example, the collagen may be cattle collagen, porcine collagen, sheep collagen, horse collagen, fish collagen, or human collagen. The type of the collagen is also not particularly limited, and may be, for example, type I collagen, type II collagen, type III collagen, type IV collagen or type V collagen.

In addition, the collagen comprised in the pharmaceutical composition of the present disclosure may be reconstituted collagen (R-collagen), which may be prepared from the collagen derived from animals. When the R-collagen is used, the environment which is more suitable for the cell growth may be provided, and thus the better eardrum regeneration may be achieved. The R-collagen may be reconstituted collagen nano particles, reconstituted collagen fibers or a combination thereof.

In some embodiments, the collagen may be type I collagen. In some embodiments, the collagen may be type I R-collagen. In some embodiments, the collagen may be type I porcine R-collagen. However, the present disclosure is not limited thereto.

The R-collagen may be prepared by the following process. The collagen (for example, type I collagen) is treated with pepsin, wherein a weight ratio of the collagen and pepsin may be in a range from 1:5 to 1:10. Then, the collagen treated with pepsin is reduced by β-mercaptoethanol to obtain single α-chain collagen. The single α-chain collagen is cross-linked by using glutaraldehyde to obtain the R-collagen, wherein the R-collagen may be a glutaraldehyde polymer-amine complex. More specifically, the R-collagen may be a glutaraldehyde collagen-amine complex. In addition, the pore size of the glutaraldehyde polymer-amine complex may be in a range from 30 μm to 150 μm, which depends upon the used amount of the glutaraldehyde.

Before treating the collagen with pepsin, the collagen may be dissolved in aqueous solution and heated to 55° C. to 65° C., followed by acidifying until the pH value is 1.8 to 2.2. In addition, after the glutaraldehyde treatment, the obtained R-collagen may be purified one or more times to remove the remaining glutaraldehyde, followed by lyophilization to obtain R-collagen powders for preparing the pharmaceutical composition of the present disclosure.

Herein, 1.8% to 2.0% (v/v) β-mercaptoethanol solution may be used in the reduction process. Furthermore, 0.5% to 25% (v/v) of glutaraldehyde may be used in the cross-linking process. The treating time of β-mercaptoethanol or glutaraldehyde is not particularly limited, and may be adjusted according to the need.

In some embodiments, the pharmaceutical composition may comprise about 19 wt % to 22 wt % of the forming agent, for example, 19 wt %, 19.5 wt %, 20 wt %, 20.5 wt %, 21 wt %, 21.5 wt % or 22 wt %. In some embodiments, the pharmaceutical composition may comprise about 19.5 wt % to 20.5 wt % of the forming agent. In some embodiments, the pharmaceutical composition may comprise about 20 wt % of the forming agent.

The forming agent used in the pharmaceutical composition is a thermosensitive or thermoresponsive polymer showing upper critical solution temperature (UCST) or lower critical solution temperature (LCST) behavior in a solvent.

For example, the polymer of PEO and PPO is a thermosensitive polymer, which becomes insoluble in water when heated due to its lower critical solution temperature (LCST). Examples for organic polymer solutions with UCST may include polystyrene in cyclohexane, polyethylene in diphenylether or polymethylmethacrylate in acetonitrile. An LCST is observed for, e.g. polypropylene in n-hexane, polystyrene in butylacetate or polymethylmethacrylate in 2-propanone. In addition, water soluble thermoresponsive materials may include poly(N-isopropylacrylamide), poly[2-(dimethylamino)ethyl methacrylate] (pDMAEMA) hydroxypropylcellulose, poly(vinylcaprolactame), poly isopropyl-2-oxazoline and polyvinyl methyl ether. However, the present disclosure is not limited thereto.

In some embodiments, the forming agent may be the polymer of PEO and PPO. In some embodiments, the forming agent may be a block copolymer of PEO and PPO. In some embodiments, the forming agent may be a PEO-PPO-PEO triblock copolymer.

In some embodiments, the forming agent may be the block copolymer of PEO and PPO with a molecular weight ranging from about 10,000 g/mol to 15,000 g/mol. In some embodiments, the forming agent may be a PEO-PPO-PEO triblock copolymer with a molecular weight ranging from about 10,000 g/mol to 15,000 g/mol. For example, the block copolymer of PEO and PPO (in particular, the PEO-PPO-PEO triblock copolymer) may have the molecular weight ranging from 10,200 g/mol to 15,000 g/mol, 10,200 g/mol to 14,800 g/mol, 10,400 g/mol to 14,800 g/mol, 10,400 g/mol to 14,600 g/mol, 10,600 g/mol to 14,600 g/mol, 10,600 g/mol to 14,400 g/mol, 10,800 g/mol to 14,400 g/mol, 10,800 g/mol to 14,200 g/mol, 11,000 g/mol to 14,200 g/mol, 11,000 g/mol to 14,000 g/mol, 11,200 g/mol to 14,000 g/mol, 11,200 g/mol to 13,800 g/mol, 11,400 g/mol to 13,800 g/mol, 11,400 g/mol to 13,600 g/mol, 11,600 g/mol to 13,600 g/mol, 11,600 g/mol to 13,400 g/mol, 11,800 g/mol to 13,400 g/mol, 11,800 g/mol to 13,200 g/mol, 12,000 g/mol to 13,200 g/mol, 12,000 g/mol to 13,000 g/mol, 12,200 g/mol to 13,000 g/mol, 12,200 g/mol to 12,800 g/mol or 12,400 g/mol to 12,800 g/mol. In some embodiments, the e block copolymer of PEO and PPO (in particular, the PEO-PPO-PEO triblock copolymer) may have the molecular weight of about 12,600 g/mol.

In some embodiments, the forming agent may be a PEO-PPO-PEO triblock copolymer represented by the following formula (I):

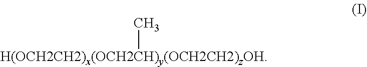

In some embodiments, x and z respectively may be an integral ranging from 85 to 110, 90 to 110, 90 to 105, 95 to 105 or 95 to 100.

In some embodiments, y may be an integral ranging from 50 to 80, 55 to 80, 55 to 75, 60 to 75, 60 to 70 or 65 to 70.

In some embodiments, the forming agent may be a PEO-PPO-PEO triblock copolymer with a molecular weight about 12,600 g/mol. In some embodiment, the forming agent may be a PEO98-PPO67-PEO98 triblock copolymer.

Except for the collagen and forming agent, the pharmaceutical composition of the present disclosure may also comprise a solvent. The solvent may comprise water, an organic solvent or a combination thereof. In some embodiments, the solvent may comprise water and acetic acid (AcOH), wherein water is the solvent for dissolving the PEO-PPO-PEO triblock copolymer, and the AcOH is the solvent for dissolving collagen. However, the present disclosure is not limited thereto, and the used solvent may be selected according to the need, for example, the type of the collagen of the forming agent.

In some embodiments, the pharmaceutical composition of the present disclosure may further comprise an additive if it is necessary. Examples of the additive may comprise antibacterial agents, active agents, adjuvants, dispersants, wetting agents, etc., for example, chitin, hyaluronic acid, alcohol, or gold or silver nanoparticles. The additive in the pharmaceutical composition is “acceptable” in the sense that it is compatible with the components of the composition and not deleterious to the subject.

In addition, the term “weight percentages” (i.e., “% by weight” and “wt %” and w/w) referenced herein, unless otherwise indicated, are based on the total weight of the pharmaceutical composition unless specified otherwise.

Furthermore, in the present disclosure, the aforesaid subject may be mammal, for example, a human, a pig, a horse, a cow, a dog, a cat, a mouse or a rat.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

The pharmaceutical composition of the present disclosure may be administered through any suitable administration rout. For example, the pharmaceutical composition may be formulated into a formulation form of ear drops or sprays. In addition, the pharmaceutical composition may be formulated into a single dosage.

For example, the pharmaceutical composition may be stored in an opaque storage bottle with a dropper or a nozzle in an aseptic manner. During administration, the pharmaceutical composition can be released to deliver to the surface of the eardrum by means of drops or sprays. In addition, a baffle may selectively be assembled on the dropper or nozzle to avoid over-projecting into the ear canal and contacting the eardrum tissue. However, the present disclosure is not limited thereto.

In some embodiments, when the pharmaceutical composition is formulated into a formulation form of ear sprays, the spray distance may be in a range from 1 mm to 10 mm, 1 mm to 9 mm, 1 mm to 8 mm, 1 mm to 7 mm, 1 mm to 6 mm, 1 mm to 5 mm, 2 mm to 5 mm or 3 mm to 5 mm. In some embodiments, the spray distance may be about 4 mm.

Other novel features of the disclosure will become more apparent from the following detailed description when taken in conjunction with the accompanying drawings.

### BRIEF DESCRIPTION OF THE DRAWINGS

FIG. 1 shows the results of gelation time test of the pharmaceutical composition of Example 1.

FIG. 2 shows the otoscope observation results before and after the surgery.

FIG. 3 shows the gross observation results of the eardrums of the experimental animals with or without the administration of the pharmaceutical composition of Example 1.

FIG. 4 shows the histopathological evaluation results of the eardrums having 50% defect of the experimental animals with or without the administration of the pharmaceutical composition of Example 1.

FIG. 5 shows the histopathological evaluation results of the eardrums having 75% defect of the experimental animals with or without the administration of the pharmaceutical composition of Example 1.

### DETAILED DESCRIPTION OF EMBODIMENT

The following embodiments when read with the accompanying drawings are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

### Preparation of R-Collagen

Type I porcine collagen was purified by the method disclosed in Einbinder J. & Schubert M., J. Biol. Chem., 188,335(1951). The purified collagen was dissolved in ddH₂O (10 mg/ml). The collagen solution was heated to 55° C. to 65° C., followed by adding HCl to obtain the collagen solution with pH 1.8˜2.2. Thus, the triple-helix of collagen can be denatured and dissolved evenly. Then, pepsin was added into the solution (in which the weight ratio of pepsin to collagen was 1:10) to remove the telopeptide at the N-terminal and the C-terminal of collagen, and the triple-helix of collagen can be converted into single α-chain collagen.

The obtained solution was treated with an alkaline solution until the pH of the solution was 10, to inhibit the activity of pepsin. Then, β-mercaptoethanol was added into the solution to reduce the remaining —S—S into —SH. Thus, the collagen can be completely converted into single α-chain collagen.

Then, glutaraldehyde (for example, 2%, 2.5% or 25%) was added into the collagen solution containing single α-chain collagen to perform the cross-linking process for 24 hours to 11 days. Glutaraldehyde can make asparagine, glutamine and arginine comprised in the collagen cross-linked to form glutaraldehyde-polymer amine complex, which is the reconstituted collagen (R-collagen).

The pH of the solution containing R-collagen was adjusted into neutral, followed by diluting with ddH₂O (1:50, v/v), precipitated by centrifugation (30 min/10,000 rpm), and removing supernatant. The aforesaid steps were performed 8 to 10 times. The obtained R-collagen was not damaged, as well as the remaining glutaraldehyde can be removed.

The obtained R-collagen was dissolved in ddH₂O (1:10, v/v), followed by slight oscillation to evenly dissolve the R-collagen. Then, the obtained solution was frozen at −70° C. for 8 hours or more, followed by lyophilization for 18˜24 hours to obtain the R-collagen powders.

### Comparative Example 1

The pharmaceutical composition of the present comparative example was prepared as follows.

3 g of the aforesaid R-collagen was dissolved in 3 ml of 5 mM AcOH. The collagen solution was heated to 40° C. for 4 hr with stirring, and the obtained R-collagen solution was stored at 4° C.

5.4 g of Pluronic F127 (F127) (PEO98-PPO67-PEO98 triblock copolymer, CAS Number 9003-11-16) was dissolved in 18.6 ml ddH₂O. The F127 solution was stirred on ice evenly, and the obtained F127 solution was stored at 4° C.

The obtained R-collagen solution was added into the obtained F127 solution at room temperature, and the mixture was stirred evenly until well mixed.

### Example 1

The pharmaceutical composition of the present example was prepared by the same process illustrated in Comparative Example 1, except that 6.0 g of F127 was dissolved in 18.0 ml ddH₂O.

### Example 2

The pharmaceutical composition of the present example was prepared by the same process illustrated in Comparative Example 1, except that 6.6 g of F127 was dissolved in 17.4 ml ddH₂O.

### Comparative Example 2

The pharmaceutical composition of the present comparative example was prepared by dissolving 5.4 g of F127 in 18.6 ml ddH₂O and stirring on ice evenly. The obtained F127 solution was stored at 4° C.

### Comparative Example 3

The pharmaceutical composition of the present comparative example was prepared by the same process illustrated in Comparative example 2, except that 6.0 g of F127 was dissolved in 18.0 ml ddH₂O.

The contents of the components in the obtained pharmaceutical compositions of Examples 1 and 2 and Comparative examples 1 to 3 are summarized in the following Table 1.

TABLE 1

| | Content | | | | |
|---|---|---|---|---|---|
| | Example | Example | Comparative | Comparative | Comparative |
| Component | 1 | 2 | Example 1 | Example 2 | Example 3 |
| F127 | 20 wt % | 22 wt % | 18 wt % | 18 wt % | 20 wt % |
| R-collagen | 10 wt % | 10 wt % | 10 wt % | — | — |

### Rheological Property Test

To determine viscosity changes of temperature versus various pharmaceutical compositions of Examples 1 and 2 and Comparative examples 1 to 3, the rheological property test was performed as follows.

2 ml samples from Examples 1 and 2 and Comparative examples 1 to 3 were respectively loaded into viscometer channels. The temperatures of the samples were gradually increased from 4° C. to 36° C. (5° C./5 min), and the viscosity of each sample was recorded at 5 min interval. The results are listed in the following Table 2.

In Table 2, “F” refers to F127 and “C” refers to R-collagen, and tri-plicate experiments were performed on the groups of Examples 1 and 2 and Comparative Example 1.

TABLE 2

| | Temperature | | | | | | | | | |
|---|---|---|---|---|---|---|---|---|---|---|
| | 4° C. | 9° C. | 14° C. | 19° C. | 24° C. | 29° C. | 34° C. | 37° C. | 39° C. | 44° C. |
| Example 1 | 142.9 | 285.7 | 1,143.3 | 1,342.0 | 6,286.0 | 10,714.0 | 11,857.0 | 11,571.0 | 11,571.0 | 11,429.0 |
| 20% F + 10% C | | | | | | | | | | |
| Example 1 | 125.0 | 250.0 | 1000.0 | 1117.0 | 5625.0 | 9500.0 | 10500.0 | 10375.0 | 10375.0 | 10250.0 |
| 20% F + 10% C | | | | | | | | | | |
| Example 1 | 100.0 | 200.0 | 900.0 | 1,000.0 | 4,500.0 | 7,800.0 | 8,900.0 | 8,600.0 | 8,600.0 | 8,500.0 |
| 20% F + 10% C | | | | | | | | | | |
| Example 2 | 142.9 | 285.7 | 428.6 | 1,571.0 | 12,571.0 | 14,857.0 | 15,000.0 | 14,714.0 | 14,571.0 | 14,571.0 |
| 22% F + 10% C | | | | | | | | | | |
| Example 2 | 125.0 | 250.0 | 500.0 | 1,375.0 | 11,375.0 | 13,375.0 | 13,500.0 | 13,125.0 | 13,000.0 | 12,875.0 |
| 22% F + 10% C | | | | | | | | | | |
| Example 2 | 100.0 | 200.0 | 400.0 | 1,100.0 | 9,300.0 | 11,100.0 | 11,300.0 | 11,000.0 | 10,800.0 | 10,800.0 |
| 22% F + 10% C | | | | | | | | | | |
| Comparative | 142.9 | 142.9 | 142.9 | 142.9 | 142.9 | 285.7 | 285.7 | 285.7 | 142.9 | 142.9 |
| Example 1 | | | | | | | | | | |
| 18% F + 10% C | | | | | | | | | | |
| Comparative | 125.0 | 125.0 | 125.0 | 125.0 | 125.0 | 250.0 | 250.0 | 250.0 | 125.0 | 125.0 |
| Example 1 | | | | | | | | | | |
| 18% F + 10% C | | | | | | | | | | |
| Comparative | 100.0 | 100.0 | 100.0 | 100.0 | 100.0 | 200.0 | 200.0 | 200.0 | 100.0 | 100.0 |
| Example 1 | | | | | | | | | | |
| 18% F + 10% C | | | | | | | | | | |
| Comparative | 83.3 | 83.3 | 83.3 | 83.3 | 166.7 | 2,583.0 | 4,167.0 | 4,417.0 | — | — |
| Example 2 | | | | | | | | | | |
| 18% F-127 | | | | | | | | | | |
| Comparative | 20.0 | 20.0 | 20.0 | 2,000.0 | 6,000.0 | 8,000.0 | 9,000.0 | 10,000.0 | 10,000.0 | — |
| Example 3 | | | | | | | | | | |
| 20% F127 | | | | | | | | | | |

As shown in Table 2, the pharmaceutical composition comprising 20 wt % or 22 wt % F127 (Examples 1 and 2) showed a stable reverse thermal property versus temperature of 35° C.˜37° C. compared to the pharmaceutical composition comprising 18 wt % F127 (Comparative Example 1). This indicates a similar temperature of the ear canal.

In addition, compared to the pharmaceutical composition comprising 22 wt % F127 (Example 2), the pharmaceutical composition comprising 20 wt % F127 (Example 1) showed a relative stable rheological property and lower variation in viscosity change at 4° C.˜14° C.

Thus, the combination of 20 wt % F127 with 10 wt % collagen (Example 1) is the optimized pharmaceutical composition that shows stable rheological and reverse thermal gelation properties to achieve an optimal gelation at the ear canal temperature.

### Gelation Time Test

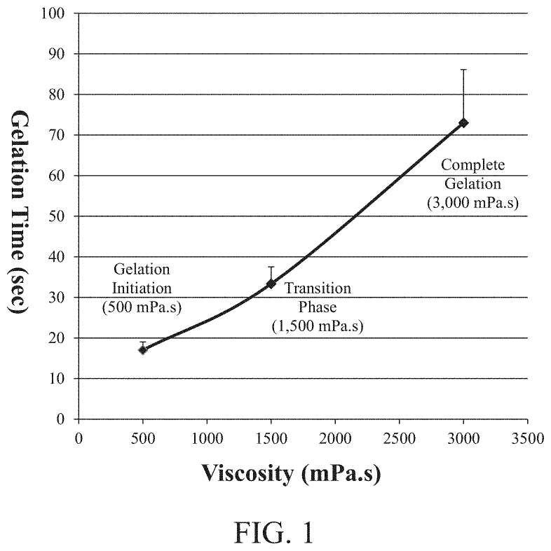

The average time of gelation phase in the pharmaceutical composition comprising 20 wt % F127 and 10 wt % collagen (Example 1) was measured at the mimic temperature of the ear canal. The results are shown in FIG. 1, which indicates that the pharmaceutical composition of Example 1 has suitable gelation time.

### Animal Model

### Efficacy Test

Male SD rats were anesthetized with intramuscular ketamine hydrochloride (40 mg/kg) and xylazine hydrochloride (5 mg/kg) before surgery. The ears of all animals were assessed using an Olympus 3-mm otomicroscope before the procedure to rule out infection. Surgery of traumatic perforation of the tympanic membrane (TM) was performed under the guiding of otoscope with a 22 # intracatheter plastic tube needle passing through the ear canals anterior and posterior to the malleus handle, in the pars tensa region of the TM. 50% marginal perforation was created on anterior part, and 75% marginal perforation was created on anterior and half posterior part.

The experimental group is set as follows:

Eardrum defect group caused by surgery:

a1) 50% marginal perforation, n=12; and

b1) 75% marginal perforation, n=12.

Eardrum defect group caused by surgery and administered with 0.3-0.5 ml of the pharmaceutical composition of Example 1:

a2) 50% marginal perforation, n=12; and

b2) 75% marginal perforation, n=12.

Half operations were performed on left side, and the other half were on the right to avoid the confounder effect.

The test endpoint is set as follows.

After the surgery, the wound healing of the eardrum was observed and recorded by using an otoscope, wherein the images of the suppuration and inflammation of the ear canal and inner ear were recorded, and the health statuses of the experimental animals were also evaluated. The observation was performed once every 3 days, 10 times, for a total of 30 days. The animals were sacrificed after the 10th observation.

After the animals were sacrificed, the eardrum tissue was excised en bloc for histological analysis. The repair of the eardrum epithelial tissue was observed, and the immune cell infiltration status of the inner ear tissue was evaluated the degree of the inflammation and suppuration.

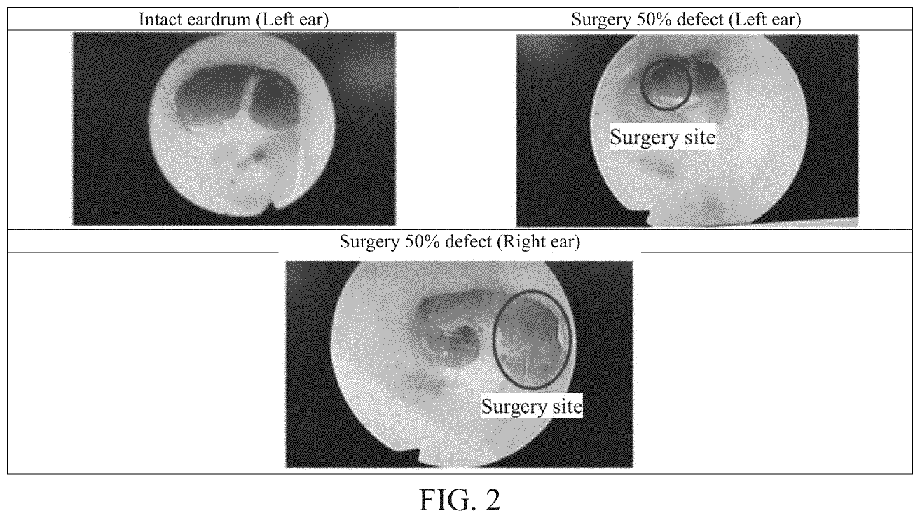

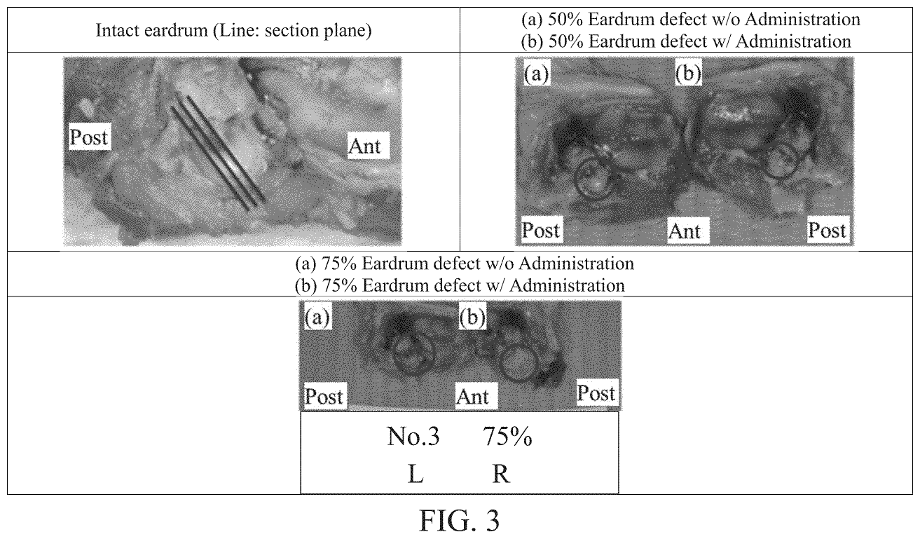

The results of the otoscope observation before and after the surgery are shown in FIG. 2, and the results of the gross observation are shown in FIG. 3. In FIG. 3, “Post” refers to the posterior part, and “Ant” refers to the anterior part. The results indicate that the eardrum defects (50% and 75% defects) can be effectively repaired when the pharmaceutical composition of Example 1 was administered to the experimental animals.

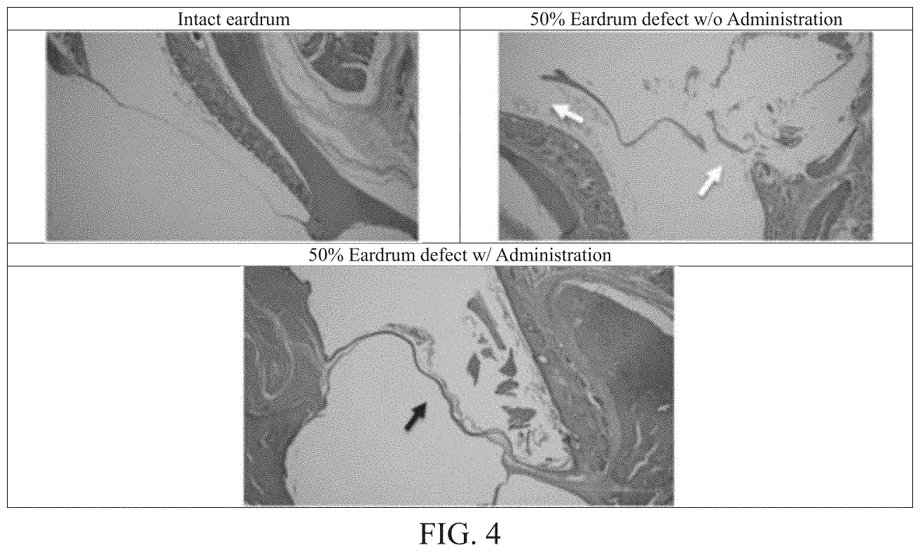

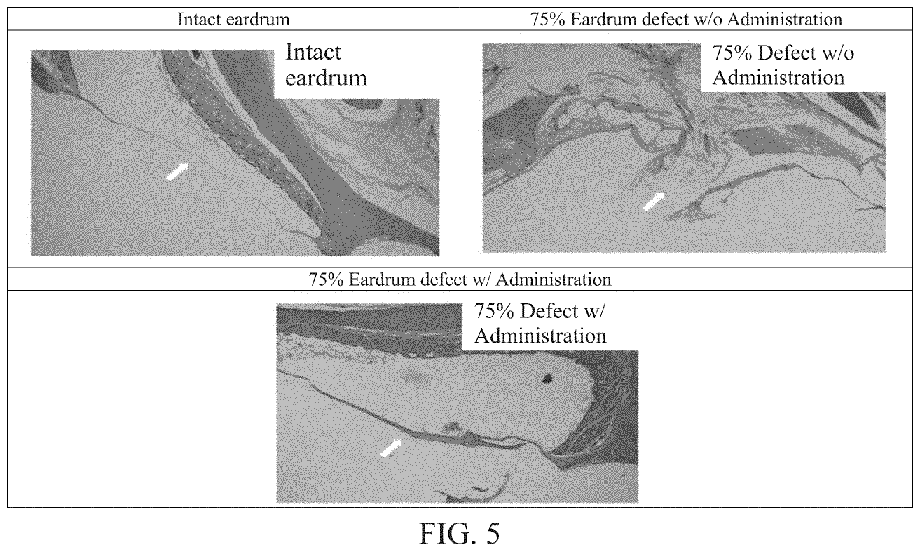

The results of the histopathological evaluation are shown in FIG. 4 and FIG. 5.

As shown in FIG. 4, under histological evaluation, the group with the administration of the pharmaceutical composition of Example 1 showed an ingrowth and recovery of the tympanic membrane tissue at the 50% defect site. In the control group without the administration of the pharmaceutical composition of Example 1, a rupture membrane structure was observed, with severe immune cell infiltration and inflammation at the adjacent tissue.

As shown in FIG. 5, the group with the administration of the pharmaceutical composition of Example 1 showed a decent membrane regeneration effect, while an intact tympanic membrane was observed without signs of inflammation in the adjacent tissue. In the control group without the administration of the pharmaceutical composition of Example 1, perforated tissue with sever fibrosis was observed at the defect site.

These results implied that the pharmaceutical composition of the present disclosure could provide a suitable tissue matrix that supports the epithelial and fibroblast cell ingrowth along the direction of the membrane thus regenerates the tympanic membrane tissue, even in the large defect case.

Although the present disclosure has been explained in relation to its embodiment, it is to be understood that many other possible modifications and variations can be made without departing from the spirit and scope of the disclosure as hereinafter claimed.

What is claimed is:

1. A pharmaceutical composition for eardrum repair, comprising:
collagen present in an amount of 8 wt % to 12 wt %;
a forming agent present in an amount of 19 wt % to 22 wt %; and
rest of a solvent,
wherein the forming agent is a polymer of polyethylene oxide (PEO) and polypropylene oxide (PPO), polystyrene, polyethylene, polypropylene, polymethylmethacrylate, poly(N-isopropylacrylamide), poly[2-(dimethylamino)ethyl methacrylate] (pDMAEMA) hydroxypropylcellulose, poly(vinylcaprolactame), poly-2-isopropyl-2-oxazoline, polyvinyl methyl ether or a combination thereof.

2. The pharmaceutical composition of claim 1, wherein the forming agent is the polymer of PEO and PPO.

3. The pharmaceutical composition of claim 2, wherein the forming agent is a block copolymer of PEO and PPO.

4. The pharmaceutical composition of claim 3, wherein the forming agent is a PEO-PPO-PEO triblock copolymer.

5. The pharmaceutical composition of claim 2, wherein the block copolymer of PEO and PPO has a molecular weight ranging from 10,000 g/mol to 15,000 g/mol.

6. The pharmaceutical composition of claim 1, wherein the collagen is type I collagen.

7. The pharmaceutical composition of claim 6, wherein the collagen is type I reconstituted collagen.

8. The pharmaceutical composition of claim 1, which has a thermo-transition temperature ranging from 32° C. to 37° C.

9. The pharmaceutical composition of claim 1, which has a gelation time less than 120 seconds.

10. A method for repairing eardrum of a subject, comprising:
administering to a subject in need thereof a pharmaceutical composition for eardrum repair, wherein the pharmaceutical composition comprises:
collagen present in an amount of 8 wt % to 12 wt %;
a forming agent present in an amount of 19 wt % to 22 wt %; and
rest of a solvent,
wherein the forming agent is a polymer of polyethylene oxide (PEO) and polypropylene oxide (PPO), polystyrene, polyethylene, polypropylene, polymethylmethacrylate, poly(N-isopropylacrylamide), poly[2-(dimethylamino)ethyl methacrylate] (pDMAEMA) hydroxypropylcellulose, poly(vinylcaprolactame), poly-2-isopropyl-2-oxazoline, polyvinyl methyl ether or a combination thereof.

11. The method of claim 10, wherein the forming agent is the polymer of PEO and PPO.

12. The method of claim 11, wherein the forming agent is a block copolymer of PEO and PPO.

13. The method of claim 12, wherein the forming agent is a PEO-PPO-PEO triblock copolymer.

14. The method of claim 11, wherein the block copolymer of PEO and PPO has a molecular weight ranging from 10,000 g/mol to 15,000 g/mol.

15. The method of claim 10, wherein the collagen is type I collagen.

16. The method of claim 15, wherein the collagen is type I reconstituted collagen.

17. The method of claim 10, wherein the pharmaceutical composition has a thermos-transition temperature ranging from 32° C. to 37° C.

18. The method of claim 10, wherein the pharmaceutical composition has a gelation time less than 120 seconds.

19. The method of claim 10, wherein the pharmaceutical composition is formulated into a formulation form of ear drops or sprays.
